(19) United States
(12) Patent Application Publication
(10) Pub. No.: US 2006/0154375 A1
(43) Pub. Date: Jul. 13, 2006

(54) Vaginal discharge evaluation kit

(76) Inventor: Kenneth E. Edelman, Solon, OH (US)

Correspondence Address: MCDONALD HOPKINS CO., LPA, 2100 BANK ONE CENTER, 600 SUPERIOR AVENUE, E., CLEVELAND, OH, 44114-2653, US

(21) Appl. No.: 11/298,546

(22) Filed: Dec. 9, 2005

Related U.S. Application Data
(60) Provisional application No. 60/634,503, filed on Dec. 9, 2004.

(51) Int. Cl. G01N 33/00

(52) U.S. Cl. 436/86

(57) ABSTRACT

A self-triage device intended for use by consumers in detecting possible yeast infections is described. The invention comprises a modified tampon wherein means for controlling the distance that the absorbent cotton member is inserted into the vagina are included. The tampon is then contacted with a pH indicating device to determine whether a possible yeast infection exists.

This application claims the benefits of U.S. Patent Application Ser. No. 60/634,503 filed on Dec. 9, 2004.

### BACKGROUND AND FIELD OF INVENTION

The use of over-the-counter antifungal medication for the treatment of presumed vulvovaginal candidiasis has become widespread over the past decade. However, the accuracy of self-diagnosis has never been validated and multiple studies have shown that self diagnosis is accurate less than 50% of the time. Moreover, the relatively high cost of such over-the-counter medications means that incorrect diagnosis and subsequent unneeded use of such medications results in unnecessary expenses to consumers in a majority of cases.

In response to this problem, various systems have been developed that help women determine when physician consultation may be more appropriate in the management of their vaginal discharge. All of these systems are premised on the fact that vaginal pH is normally less than or equal to 4½ and the fact that not all vaginal discharge is due to an underlying infection. In fact, if the pH of vaginal discharge is found to be 4½ or less this could be a normal bodily function, a yeast infection or potentially a sexually transmitted disease. However, if the vaginal pH is greater than 4½ this could be due to a bacterial infection such as bacterial vaginosis or a sexually transmitted disease such as trichomonas. Other factors could also cause the vaginal pH to become greater that 4½ such as blood, cervical mucus, vaginal lubricants, semen, and recent douching. Consequently, these previous systems utilize a simple pH test whereby a sample of discharge is pH tested and a pH greater than 4½ is used as indicative of the need for further consultation with a doctor.

In order to accomplish this evaluation of vaginal discharge effectively, a sample must be obtained directly from the lower portion of the vagina. In the ideal clinical setting, this sample is obtained in the office with the use of a speculum to allow direct visualization of the vagina. Such visualization permits the specimen to be accurately/strategically obtained from the lower portion of the vagina (the portion closest to the vulva) avoiding the cervical mucus. Once the specimen has been obtained, usually with a long cotton swab, the sample is then placed on pH paper and using the color code the correct pH can be determined. Accurate sampling of vaginal discharge can be very difficult without having to perform a pelvic exam and without having to use a speculum for direct visualization of the correct sample being obtained.

In contrast, if the sample is obtained by the use of a pad that is placed outside of the vagina, the discharge can be contaminated by the patient's sweat, body lotions, laundry detergents and skin secretions. In addition, if the patient is not having a lot of discharge, it could take a long time to obtain a sample for evaluation.

Consequently, a safe, easy-to-use device would be welcome. Specifically, such a device must not cause or otherwise allow for the possibility of self-inflicted injury by a user who is not a trained gynecologist, thereby excluding systems that rely on a consumer inserting a thin swab, plastic stick or other narrow or pointed object into the vagina. Also, the device must be easy to use in order to consistently and accurately obtain a sample from the lower portion of the vagina. The device should also be familiar to consumers so that it can be used easily with minimal discomfort.

### SUMMARY OF INVENTION

An evaluation kit is based on testing the pH of vaginal discharge. This system relies on modified, commercially available tampons used in conjunction with a pH indicator, such as pH paper or a pH monitor, in order to allow for a quick, preliminary analysis and self-triage of vaginal discharge that is suspected to be a yeast infection.

In one embodiment, the tampon is one continuous diameter and contained entirely within an insertion tube which itself has markings or physical stops to prevent full expulsion of the tampon from the insertion tube. Here, the tampon is used in conjunction with a pH indicator, either provided integrally with the tampon or separately, to create a self-triage kit. In another embodiment, a self-triage method is contemplated which relies upon the pH testing of vaginal discharge comprising inserting a non-lubricated tampon having an absorbent member contained within an applicator tube a distance into a vagina relative to the applicator tube determined by guide markings on the applicator tube or absorbent member, collecting fluid on the absorbent member for a time less than 1 minute and immediately exposing the absorbent member to a pH indicator.

### BRIEF DESCRIPTION OF THE DRAWINGS

FIG. 1 shows an illustration of the invention.

### DESCRIPTION OF PREFERRED EMBODIMENTS

The present invention comprises a modified non lubricated cotton tampon. This particular tampon has an outer insertion portion (cardboard or plastic applicator) and an inner plunger that allows a portion of the cotton tampon to be released into the vagina. The tampon/applicator is modified in the following way to ensure that when the device is placed into the vagina the specimen will be obtained from the lower portion of the vagina:
1. The inner plunger is shortened in length to allow only ¼ to ⅓ of the tampon to be exposed when the plunger is fully inserted into the outer portion of the applicator. This prevents the tampon from being released into the vagina. The tampon may be affixed to the shortened plunger to insure that the tampon itself is not released from the applicator.
2. The tampon must fit securely in the insertion device even when partially expelled. This will allow the tampon to not separate when the device is removed from the vagina. In one embodiment, this contemplates a tampon having a consistent diameter along its entire length whereby the diameter closely matches that of the applicator.
3. The insertion portion of the tampon has a marker/raised edge/and or groove or other physical stop approximately 3 cm from the edge. This will mark the depth of insertion of the collection device into the vagina. This stopping means causes the patient to insert the applicator into the vagina only to the marker/groove/raised stopper and then depress the plunger which will advance a portion of the cotton tampon into the vagina allowing for accurate sampling of the lower portion of the vagina.

An individual using this specific specimen collector would insert the device into the vagina to the level of the indicator. The next step would be to slowly press the plunger device advancing the tampon into the proximal portion of the vagina. Because of the above mentioned modification, the specimen collector would now be able to obtain a sample of the vaginal discharge from the lower portion of the vagina avoiding potential contamination from the cervix. The device is then removed from the vagina a few seconds after the plunger has been advanced, and the exposure time should be no more than a few minutes in order to avoid potential contamination of the sample. All components are removed when the device is removed from the vagina. When the device is removed the exposed portion of the tampon should now contain a sample of the vaginal discharge. The sample is now ready to be immediately placed onto the pH indicator portion of the kit.

The exact diameter of the tampon or the insertion device does not matter, although consideration should be given to avoid using too small of a diameter which might puncture the skin. Most important is that when the modifications that are described above are performed to any tampon the final product will be able to reproducibly obtain a sample from the lower portion of the vagina. The exact location of the marker/groove/raised area on the insertion portion of the device and the length of tampon that is exposed will be calculated based on the product size to allow for a combined length of 4-5 cm. This modification should avoid possible cervical contamination and allow enough length to safely place the device far enough into the vagina to avoid contamination from the vulva/perineum.

The pH indicator comprises the second significant aspect of the invention. This portion of the vaginal discharge evaluation kit is comprised of a modified plastic compact. Instead of there being makeup or a mirror inside there would be a centrally placed piece of pH paper. The paper would be larger in size than the tip of the specimen collector. This pH paper would change color when the pH is greater than 4½. After the vaginal discharge sample has been obtained as detailed above, the lid of the indicator kit is opened and the cotton tip portion of the applicator is immediately placed on the pH paper. The color key for the results can be placed around the ph paper. The upper half can be the color for normal vaginal pH which is less than or equal to 4½. This would have markings saying this a normal pH or possible yeast infection. The lower portion of the kit would demonstrate all other pH values which would be marked as abnormal vaginal pH requiring possible attention from a trained physician.

The exact color of the positive control indicating a pH of greater than 4½ will be determined by which brand of pH paper is used in the indicator device. Another key factor in selecting the pH indicator will be the cost, as one of the primary benefits of the present invention is its low cost and ease of use. Ultimately, an indicator with a narrow color change band at or around 4½ is preferred, although multi-colored full range papers would also be appropriate.

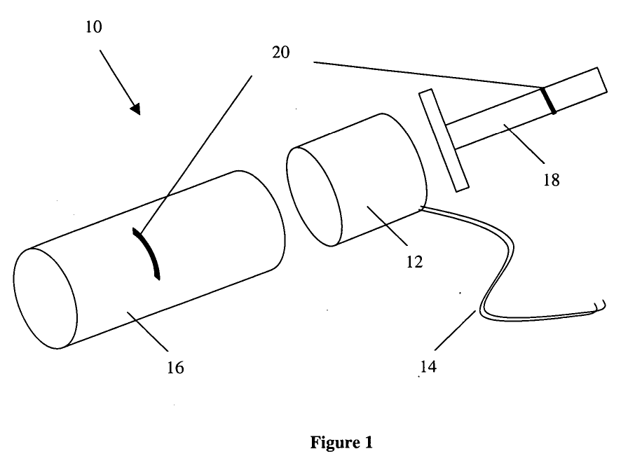

The invention itself is shown in FIG. 1. Notably, this expanded perspective view is not necessarily drawn to scale, and the dimensions referenced above are more representative of the scope of the invention. By the same token, modifications can be made to those stated dimensions without departing from the inventive principle contemplated herein.

Modified tampon 10 includes an absorbent member, such as a non-lubricated cotton bolt 12 with optional safety removal string 14 affixed to one end of the bolt 12. The bolt 12 is encased within an applicator tube 16, and plunger 18 encloses the tube 16 at one end while the other end of tube 16 provides an opening through which the cotton bolt 12 passes so as to be exposed to vaginal discharge within the lower portion of the vagina. Although bolt 12 should not be completely expelled from the tube 16 during the operation of the invention (thereby allowing the user to safely and easily handle the tube 16 during self-triage testing according to the present invention), optional safety string 14 may protrude thru the plunger-end of tube 16 so as to permit the bolt to be removed from the vagina in the event the bolt 12 is pushed too far into the vagina to be removed with the applicator tube 16. String 14 may be replaced by any other implement that allows the user to remove the bolt 12 in the event the bolt is accidentally released from the tube 16.

Applicator tube 16 is modified to insure that the bolt 12 is not expelled too far into the vagina through the inclusion of means for controlling the distance which the bolt is released into the lower portion of the vagina. As discussed above, if a sample is not obtained from the lower portion of the vagina, pH testing to determine the presence of a yeast infection may not be accurate. The modification can be in the form of means which prevent the plunger from being fully deployed, such as a marking 20 on the exterior of the applicator tube 16 and/or plunger which indicates to the user how far to push the plunger. Alternatively, the bolt 12 may be shortened, the tube 16 lengthened beyond the distance which the plunger 18 can be deployed, a viewing window or slit may be made in the tube to see how far the plunger is depressed and/or the tube 16 may further include an interior ring or other stopping mechanism to prevent pushing the plunger 18 beyond a certain distance.

Bolt 12 should be contacted with fluid in the lower vagina for a sufficient amount of time to permit the discharge adhere to the bolt 12, typically achieved in a matter of seconds. Notably, because the means for preventing controlling the distance can take the form of a marking on the applicator tube 16 or the lengthening of the tube 16 itself, the user can manipulate tampon 10 via tube 16 so as to avoid contact with the cotton bolt 12, which reduces the risk of contaminating the sample through incidental contact with the user's skin and/or the upper portion of the user's vagina.

The plunger 18 can be integrally formed and connected to the bolt 12 so as to form a single unitary piece. Alternatively, the plunger can be omitted in its entirety so long as bolt 12 is sufficiently long enough to allow the user to control how far the bolt is expelled from the tube 16.

The entire invention may be packaged so as to allow for storage of the tampon 10 in a device which also integrates the pH indicator. A preferred means of integrating these elements of the self-triage test kit is to provide pH paper and an appropriate color comparison chart directly on the packaging of the tampon itself. Such paper could also be incorporated into the instruction pamphlet included with the kit or via other known means.

The invention may be implemented using currently available commercial materials. In particular, ordinary tampons can be marked on the exterior with a sticker to provide a guide to the user and to act as the means for controlling the distance which plunger is inserted into the vagina. This arrangement would insure that minimal additional costs would be incurred by a prospective manufacturer of the device.

This invention will find further utility in the low cost of its materials and compact size. As such, it is anticipated that the invention could be sold with the aforementioned over-the-counter yeast infection medications as a consumer value-added element for that product. That is, through the concurrent use of the invention with such medications, consumers can reduce or avoid altogether the unnecessary use of such medications.

While a specific embodiment has been described in detail above, it should be understood that the inventor contemplates and intends for all known equivalent materials and elements to be encompassed by this disclosure without departing from the inventive principle stated herein.

I claim:

1. A self-triage kit for evaluating vaginal discharge comprising:
an absorbent member having a constant diameter;
an applicator at least partially enclosing the absorbent member, said applicator positioned proximate to a user's vagina;
means for controllably expelling a portion the absorbent member to allow for collection of fluid from a lower portion of the user's vagina; and
a pH indicator contacted with fluid collected from the lower portion of the user's vagina.

2. The self-triage kit according to claim 1, further comprising a removal device attached to the absorbent member.

3. The self-triage kit according to claim 1, wherein the pH indicator distinguishes pH levels greater than 4½.

4. A self-triage kit for evaluating vaginal discharge comprising:
an applicator tube having a terminal opening and a selected constant diameter;
a cylindrical absorbent member substantially conforming to the selected diameter of the applicator tube, said absorbent member carried within the applicator tube;
means for controllably expelling a portion of the absorbent member through the terminal opening; and
a pH indicator.

5. The self-triage kit according to claim 4, further comprising a removal device attached to the absorbent member.

6. The self-triage kit according to claim 4, wherein the pH indicator distinguishes pH levels greater than 4½.
